(19) United States
(12) Patent Application Publication
(10) Pub. No.: US 2022/0202819 A1
(43) Pub. Date: Jun. 30, 2022

(54) USE OF ANTIPSYCHOTIC SUBSTANCES FOR TREATMENT OF INFECTIOUS DISEASES

(71) Applicant: Claire E. Pratty, Providence, RI (US)

(72) Inventor: Claire E. Pratty, Providence, RI (US)

(21) Appl. No.: 17/139,379

(22) Filed: Dec. 31, 2020

(51) Int. Cl. A61K 31/519; A61K 31/554; A61K 31/496; A61K 31/5513; A61K 31/407; A61K 31/4468; A61K 31/538; A61K 31/5415; A61K 31/454

(52) U.S. Cl. CPC A61K 31/519; A61K 31/554; A61K 31/496; A61K 31/5513; A61K 31/454; A61K 31/4468; A61K 31/538; A61K 31/5415; A61K 31/407

(57) ABSTRACT

Methods for treatment and prevention of infectious disease are described. Such infectious diseases may include, for instance, viruses, novel viruses, coronaviruses, and the like. Procedures and protocols for use of various medications, such as typical or atypical antipsychotic medications, to prevent and/or treat infection diseases are presented. Such medications include substances that act as receptor antagonists, partial antagonists, agonists, and/or partial agonists. The medications may specifically include antagonists to dopamine receptors including but not limited to dopamine-2 (D2) receptors and/or serotonin receptors including but not limited to hydroxy-tryptamine receptors.

### BACKGROUND

Infectious diseases adversely affect many people. Many such people and/or organizations (e.g., employers, government bodies, etc.) may wish to reduce the effects of such diseases by limiting spread and/or severity of symptoms.

### BRIEF DESCRIPTION OF THE SEVERAL VIEWS OF THE DRAWINGS

The novel features of the disclosure are set forth in the appended claims. However, for purpose of explanation, several embodiments are illustrated in the following drawings.

FIG. 1A illustrates a dosing schedule according to an exemplary embodiment;

FIG. 1B illustrates an evaluation schedule according to an exemplary embodiment;

FIG. 2A-FIG. 2C illustrate receptor activation by an infectious disease;

FIG. 3A-FIG. 3B illustrate use of antagonist substances to prevent or mitigate effects of pathogens according to an exemplary embodiment; and

FIG. 4 illustrates a plot of results using a treatment protocol of some embodiments.

### DETAILED DESCRIPTION

The following detailed description describes currently contemplated modes of carrying out exemplary embodiments. The description is not to be taken in a limiting sense, but is made merely for the purpose of illustrating the general principles of some embodiments, as the scope of the disclosure is best defined by the appended claims.

Various features are described below that can each be used independently of one another or in combination with other features. Broadly, some embodiments generally provide methods for treatment and prevention of infectious disease.

Examples of such infectious disease include, for instance, viruses, novel viruses, coronaviruses, single-stranded ribonucleic acid (RNA) viruses, and the like. Throughout this disclosure, the terms “infectious disease”, “virus”, “coronavirus”, “COVID” (coronavirus disease) or “COVID”, “SARS-CoV” (severe acute respiratory syndrome coronavirus), middle east respiratory syndrome-related coronavirus (MERS-CoV), and/or similar terms may be used to refer to any applicable infectious diseases or species of disease (e.g., coronavirus), not limited to a specific strain or type of infectious disease. Such terms may include other identifying or differentiating information, such as a year (e.g., “COVID-19”), version (e.g., “SARS-CoV-2”), originating region, etc.

Those with conditions associated with treatment using antipsychotic medications (e.g., schizophrenia, bipolar disorder, major depressive disorder, schizoaffective disorder, dementia, etc.) would typically be at higher risk of contracting an infectious disease or suffering from more severe symptoms than others. For instance, many people treated with such conditions may be likely to have comorbidities such as diabetes, heart disease, obesity, etc. Further, such people may be more likely to use controlled substances. In addition, those with conditions such as schizophrenia or bipolar disorder may be more likely to live in group facilities and share resources such as bathrooms or otherwise touch common fixtures, items, or equipment, thus leading to spread of contagious disease. However, as discussed in more detail below, such populations appear, relative to the general population, to be more resistant to contacting COVID-19 and have less severe symptoms if they do contract the disease. Such a result is the opposite of what would be expected of such a high-risk population.

Some embodiments may include procedures or protocols for use of various substances (also referred to as “medications”, “medicaments”, “substances”, and the like), such as typical antipsychotic substances (also referred to as “first generation” antipsychotics or “FGAs”) and/or atypical antipsychotic substances (also referred to as “second generation” antipsychotics or “SGAs”), to prevent and/or treat infectious diseases such as COVID-19

Throughout this disclosure, references to “antipsychotic” substances may include FGAs, SGAs, typical antipsychotics, atypical antipsychotics, and/or other similar medications or substances, as described below. Such antipsychotic substances may generally include substances that reduce dopaminergic neurotransmission by acting on one or more dopamine receptors (e.g., a dopamine-2 or “D2” receptor, “D1” receptor, “D3” receptor, “D4” receptor, “Dₙ” receptor, etc.) and/or one or more serotonin receptors (e.g., a 5-hydroxy-tryptamine or “5-HT2A” receptor, “5-HT1A” receptor, etc.).

Although specific examples above and below may reference atypical antipsychotic medications, typical antipsychotic medications, FGAs, SGAs, and/or specific examples of such substances, one of ordinary skill in the art will recognize that such description may be applied to various other types of antipsychotic substances, other types of substances that may block various receptors associated with transmission of infectious disease, and/or other specific examples of such substances, as described below.

FGAs act as dopamine receptor antagonists, and specifically antagonists to D2 receptors. The pharmacodynamic profile of SGAs is characterized by rapid D2 dissociation, 5-HT2A antagonism, and 5-HT1A agonism. Throughout this disclosure, the terms “atypical antipsychotic medications” (and/or specific examples of such medications), “typical antipsychotic medications” (and/or specific examples of such medications), “D2 blocker”, and/or other similar terms, may be used to refer to any appropriate receptor antagonist, partial antagonist, agonist, and/or partial agonist.

A process associated with prophylaxis may include administration of a low dose, very low dose, or “subtherapeutic” dose of an antipsychotic medication, where subtherapeutic refers to use for treatment of mental illness or related conditions rather than use for prevention of infectious disease. As one example, aripiprazole may be prescribed for an otherwise healthy adult who is not taking other prescription medications as (1) aripiprazole tablets at a dose of one to two milligrams per day (e.g., one milligram per twenty-four hour period or two milligrams per twenty-four hour period) or as (2) aripiprazole long acting injections (LAI) at a dose of three hundred to four hundred milligrams per month.

A process associated with treatment may similarly include administration of a low dose, very low dose, or subtherapeutic dose of an antipsychotic substance, where subtherapeutic refers to use for treatment of mental illness or related conditions rather than use for treatment of infectious disease. As one example, aripiprazole may be prescribed for an otherwise healthy adult who is not taking other prescription medications as (1) aripiprazole tablets at a dose of one to two milligrams per day (i.e., one milligram per twenty-four hour period or two milligrams per twenty-four hour period) or as (2) aripiprazole long acting injections (LAI) at a dose of three hundred to four hundred milligrams per month.

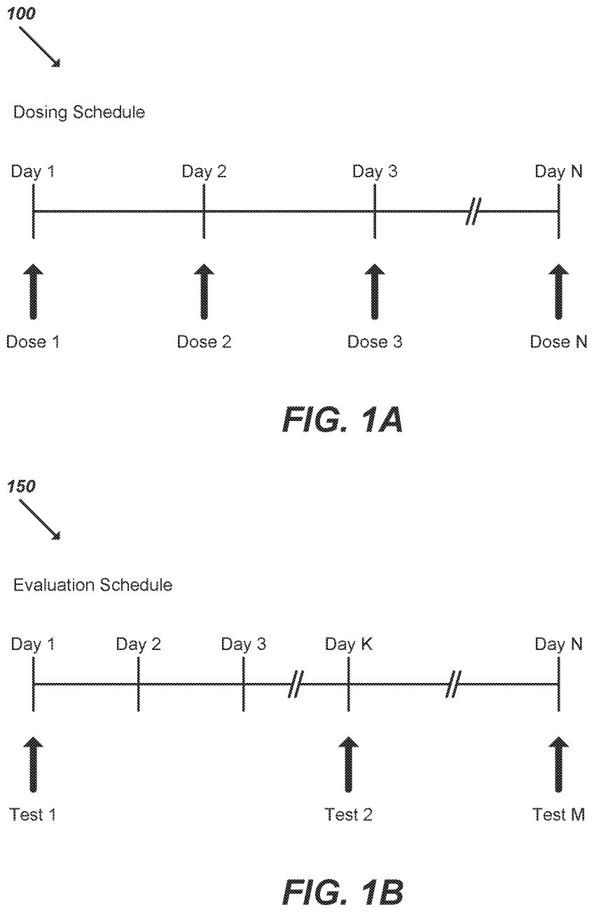

FIG. 1A illustrates a dosing schedule 100 according to an exemplary embodiment. As described above, some embodiments may include low-dose use of antipsychotic substances to prevent or treat infection diseases. Such a dosing schedule may be recommended for various subjects (also referred to as “patients”, “entrants”, etc.).

Because of the low risks of side effects associated with low dose use, the dosing schedule may be recommended for various individuals and/or populations based on risk profile or anticipated risk profile, availability of medications, and/or other relevant factors. For instance, in regions where the medications are widely available and the population routinely complies with instructions, the treatment and prophylaxis regimens described herein may be recommended for all adults. In some cases, medications may be specified for prophylaxis for those at increased risk (either through priority or implementation). Examples of populations at higher risk may include, for instance, the elderly, those with comorbidities such as heart disease, obesity, or diabetes, individuals with lung problems, cancer patients, individuals with reduced immunity, individuals with developmental disabilities, and/or other groups.

Some populations may be at higher risk due to environmental factors. For instance, residents of congregate living facilities such as nursing homes, skilled nursing facilities, detention centers and institutions, prisons, boarding houses, group homes, recovery houses, homeless shelters, and/or those in similar situations may be at higher risk of contracting infectious diseases. Further, specific individuals or groups of individuals (such as the other residents of a household, coworkers, etc.) may be at higher risk if a close contact has contracted a disease (or received a positive test diagnosis).

Some populations may be at higher risk due to factors such as habitual and/or social determinants of health. For example, some individuals may be at greater risk due to income and social status, availability and/or quality of social support networks, employment and/or working conditions, financial instability, social environment, physical environment, hygiene, personal health practices and coping skills, cultural practices, and/or other such factors. Furthermore, such adversities may present greater risk not only to a specific individual, but to all members within a household, particularly if a household is limited to shared living quarters and unable to quarantine and/or isolate upon illness onset or contact with a carrier of the infectious disease and/or other exposure.

Other populations at high risk may include essential workers, particularly healthcare workers who have direct contact with their patients and/or those working in close proximity to other people. Specific determinations may be made by individual practitioners and individual patients based on personal risk factors, social determinants of health, comorbidities, other medications the patient may already be taking, and/or other relevant factors. Risk factors may be evaluated over time. For instance, other members of a household may be prescribed a treatment after an initial diagnosis but may stop treatment after some specified duration or some other criteria (e.g., a negative test result).

In this example, the dosing schedule 100 (also referred to as a “regimen”, “prescription”, “routine”, etc.) includes a single dose (e.g., a one to two milligram tablet of aripiprazole) taken daily for some specified number of days. Different embodiments may include different dosing schedules that may include differing delays between administration of doses. For instance, a dose may be prescribed two times per day or a dose may be prescribed every other day. As another example, a dosing schedule may include various “off days” where no dose is specified. Example intervals may include daily, as shown, weekly, bi-weekly, monthly, quarterly, etc. and/or any other periodic irregular intervals.

In some cases, the intervals may vary across schedule 100 or due to various conditions. For instance, a medication may be changed from an oral or ingestible type (e.g., tablets) to an injectable form and a portion of the dosing schedule 100 may thus include daily dosing, as appropriate for tablets, and another portion of the dosing schedule 100 may include monthly dosing, as appropriate for an injectable type dose. Dosing schedule 100 may be varied due to changes in medication form or type, availability of medication, and/or other relevant factors (e.g., onset of adverse side-effects, change in status, etc.).

The route of medication administration may be changed based on bioavailability, pharmacokinetic data, safety and efficacy data, and/or other relevant factors. The medication administration route may be varied among any number of available routes (and/or types or sub-types of medications) including, but not limited to, ingestible forms, extended-release forms, sublingual forms, injectable forms, inhaled forms, and/or other appropriate forms or routes.

The dose amount may vary during schedule 100 in some cases. For instance, some embodiments may prescribe an increased initial dose (or doses) that tapers up (or down) to a maintenance amount. For instance, the dose amount from an initial dose of one milligram for one or two days (or other initiation period) and continuing with a one to two milligram daily dose after the initiation period). As another example, dose amount may be adjusted over time based on measured data (e.g., viral load) or other relevant factors. As still another example, a schedule may include various “skip” days where no dose is specified. Further, the dosing schedule and/or amount may vary due to a patient and/or practitioner's administration of a prescription (e.g., a patient may forget a dose one day or miss a dose due to travel).

In this disclosure, “low-dose” may include doses lower than those typically prescribed for other conditions and/or indicate a lowest available measure of a substance (e.g., a minimum available tablet size). For instance, continuing the aripiprazole example, the substance may typically be prescribed in amounts such as twenty to thirty milligrams per day for schizophrenia and fifteen to thirty milligrams per day for bipolar disorder. As another example, aripiprazole may be prescribed in amounts between two and five milligrams per day in conjunction with anti-depressant medication. By prescribing at lower than typical doses, undesirable side effects, such as headaches, weight gain, lethargy, and daytime sedation may be reduced or eliminated. Table 1, below, lists several examples of low-dose (or subtherapeutic dose) atypical antipsychotic substances that may be applicable for preventing and/or treating infectious disease.

TABLE 1

| | Typical Dose Amount | Low-Dose Amount |
|---|---|---|
| Substance | (Antipsychotic) | (Infectious Disease) |
| aripiprazole | 5-30 mg/day (tablet or orally | 1-5 mg/day (tablet or orally |
| | disintegrating tablet) | disintegrating tablet) |
| | 300-400 mg/month (long-acting | 300 mg/month (long-acting |
| | injectable) | injectable) |
| | 442-1064 mg/1-4 months (long- | 442 mg/month (long-acting |
| | acting injectable) | injectable) |
| brexpiprazole | 2-4 mg/day (tablet) | 0.25-2 mg/day (tablet) |
| olanzapine | 5-40 mg/day (tablet or orally | 2.5-5 mg/day (tablet or orally |
| | disintegrating tablet) | disintegrating tablet) |
| quetiapine, | 150-800 mg/day (tablet or | 25-75 mg/day (tablet) |
| quetiapine fumarate | extended-release tablet) | 50 mg/day (extended-release |
| | | tablet) |
| paliperidone palmitate | 1.5-9 mg/day (tablet or extended- | 1.5 mg/day (tablet or extended- |
| | release tablet) | release tablet) |
| | 156-234 mg/month | 117 mg/month (intramuscular |
| | (intramuscular injectable) | injectable) |
| | 546-819 mg/3 months | 237-410 mg/3 months |
| | (intramuscular injectable) | (intramuscular injectable) |
| risperidone | 1-8 mg/day (tablet or orally | 0.25-1 mg/day (tablet or orally |
| | disintegrating tablet) | disintegrating tablet) |
| | 25-50 mg/2 weeks (long-acting | 12.5-25 mg/2 weeks (long- |
| | injectable) | acting injectable) |
| | 50-100 mg/month (long-acting | 25-50 mg/month (long-acting |
| | injectable) | injectable) |
| ziprasidone HCl | 20-160 mg/day (tablet) | 20 mg/day (tablet) |
| cariprazine HCl | 1.5-6 mg/day (tablet) | 1.5 mg/day (tablet) |
| asenapine | 5-20 mg/day (tablet) | 5 mg/day (tablet) |
| pimavanserin | 34 mg/day (tablet) | 34 mg/day (tablet) |
| lurasidone HCl | 40-160 mg/day (tablet) | 20 mg/day (tablet) |
| iloperidone | 8-24 mg/day (tablet) | 4-8 mg/day (tablet) |

Table 2, below, lists several examples of low-dose (or subtherapeutic dose) typical antipsychotic substances that may be applicable for preventing and/or treating infectious disease.

TABLE 2

| | Typical Dose Amount | Low-Dose Amount |
|---|---|---|
| Substance | (Antipsychotic) | (Infectious Disease) |
| trifluoperazine HCl | 5-60 mg/day (tablet) | 1-5 mg/day (tablet) |
| thiothixene | 5-60 mg/day (tablet) | 1-5 mg/day (tablet) |
| perphenazine | 8-64 mg/day (tablet) | 2-8 mg/day (tablet) |
| loxapine | 10-100 mg/day (tablet) | 5 mg/day (tablet) |
| haloperidol, | 5-50 mg/day (tablet) | 0.5-2 mg/day (tablet) |
| haloperidol lactate | | |
| haloperidol decanoate | 25-150 mg/month (intramuscular | 25 mg/month (intramuscular |
| | injectable) | injectable) |
| fluphenazine HCl | 5-60 mg/day (tablet) | 1-5 mg/day (tablet) |
| fluphenazine decanoate | 25-50 mg/2 weeks (intramuscular | 25 mg/month (intramuscular |
| | injectable) | injectable) |
| chlorpromazine HCl | 100-1000 mg/day (tablet) | 10-50 mg/day (tablet) |

Different embodiments may be implemented with various different specific substances (and/or combinations of substances as used in “adjunct” or “combination” therapy) and/or associated dosage, as described herein. Thus, for example, any of the above-referenced typical or atypical antipsychotics may be combined with one or more other typical and/or atypical antipsychotics. In addition, although various specific forms (e.g., tablets, pills, capsules, elixir, etc.) or sub-forms (e.g., orally disintegrating tablets, extended release tablets, etc.), methods of administration (e.g., orally, injection, etc.), dosing ranges, and/or dosing schedules (e.g., daily, weekly, biweekly, monthly, trimonthly, quarterly, etc.) may be described, one of ordinary skill in the art will recognize that the approaches described herein may be applied to various different forms or sub-forms, methods of administration, dosing ranges, and/or dosing schedules, as appropriate or available.

FIG. 1B illustrates an evaluation schedule 150 according to an exemplary embodiment. Such an evaluation schedule may be associated with various collected data and evaluations of efficacy. For instance, such an evaluation schedule may be applied to various groups to determine effective dosages.

In this example, the evaluation schedule 150 is complementary to the dosing schedule 100. In other words, evaluation schedule 150 may be associated with daily dosing.

The example evaluation schedule includes an initial test on the first day, followed by periodic tests every k days (e.g., weekly or biweekly testing) for the duration of the dosing schedule 100 (i.e., until day N in this example).

Each test or evaluation may include, for instance, a binary test or metric (e.g., a test for an active virus with a positive or negative result). Each test or evaluation may include various scored or relative metrics. For instance, a viral load test may include a result or value such as a normalized value from zero to one hundred, a particle ratio (e.g., parts per million), and/or other appropriate metrics indicating the test result.

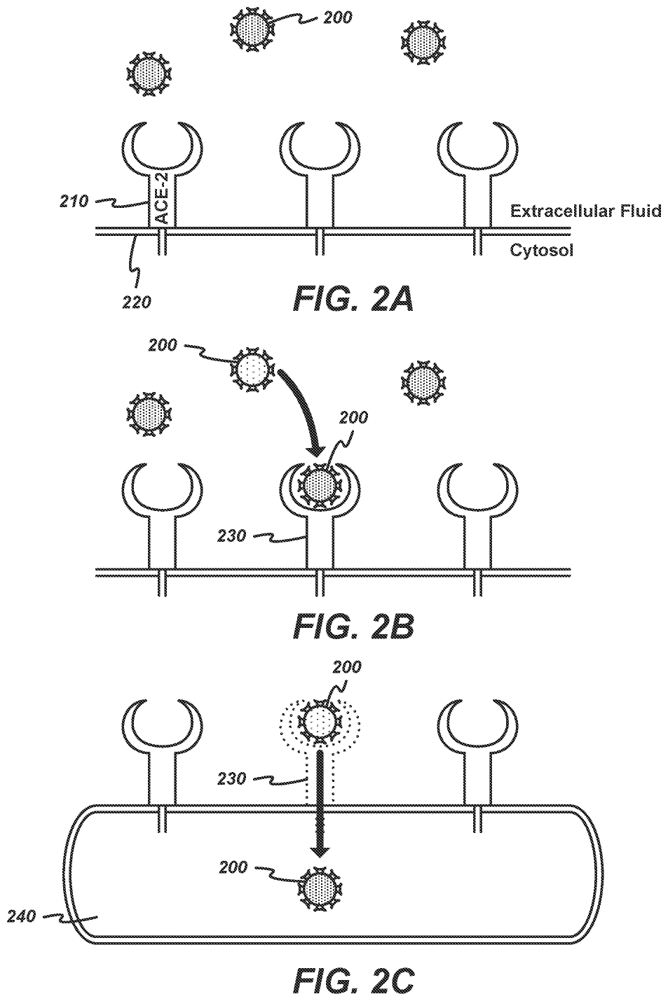

FIG. 2A illustrates a portion of the renin-angiotensin system (RAS). One of ordinary skill in the art will recognize that FIG. 2A is (and similar figures are) a stylized representation of various biological components or particles that is presented for clarity and concision and may not accurately depict particle sizes, structures, geometries, etc.

Host immune dysfunction caused by infectious disease such as COVID-19 may involve the RAS and angiotensin-converting enzymes (ACE) receptors, specifically the ACE-2 receptor. In this example, various infectious disease particles 200, such as virus molecules or other particles or structures may be suspended in extracellular fluid.

A particular portion of a cell may include, as shown, various receptors 210, such as ACE-2 receptors or other D-2 receptors along plasma membrane 220, which separates the cytosol (or intracellular fluid) from extracellular fluid.

FIG. 2B illustrates occupation of a receptor 210 by a disease particle 200. As shown, disease particle 200 has moved from an initial location (indicated by a lighter fill pattern) to a location associated with receptor 230. FIG. 2C illustrates endocytosis of disease particle 200 by receptor 230, where the particle 200 has been absorbed into the cell body 240 via receptor 230.

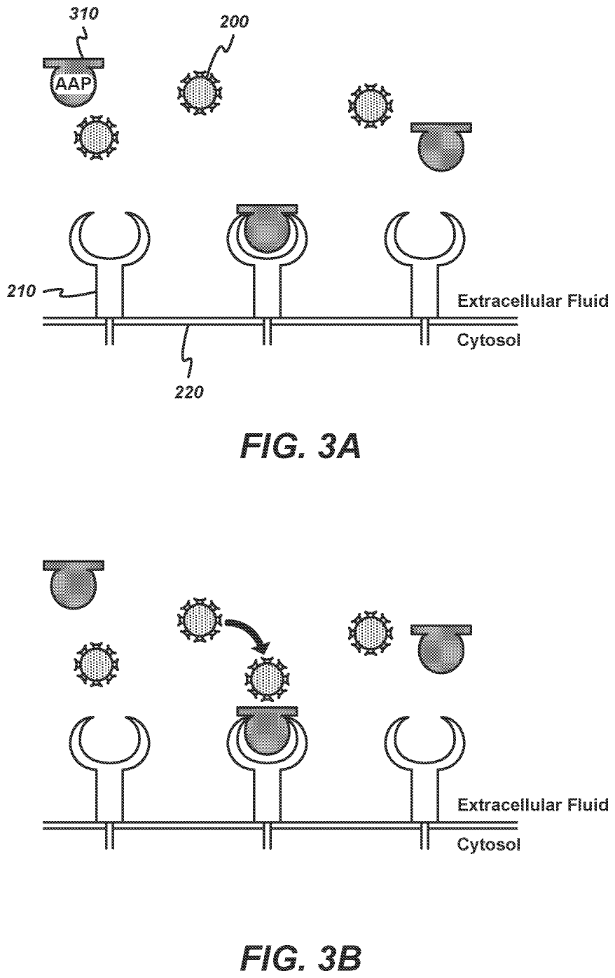

FIG. 3A illustrates use of antagonist substances to prevent or mitigate effects of pathogens according to an exemplary embodiment. As shown, various antipsychotic substance particles and/or other antagonist substances 310 may attach to one or more receptors 210.

FIG. 3B illustrates blocking of a receptor 210 by an antagonist substance 310. The viral particle 200 is prevented from being absorbed at the receptor 210 by the antagonist substance 310. In this way, the antagonist substance 310 may prevent at least some of the viral particles 200 (and/or other infectious disease particles) from being absorbed into the cells, thus mitigating or eliminating effects of the disease associated with the particles 200.

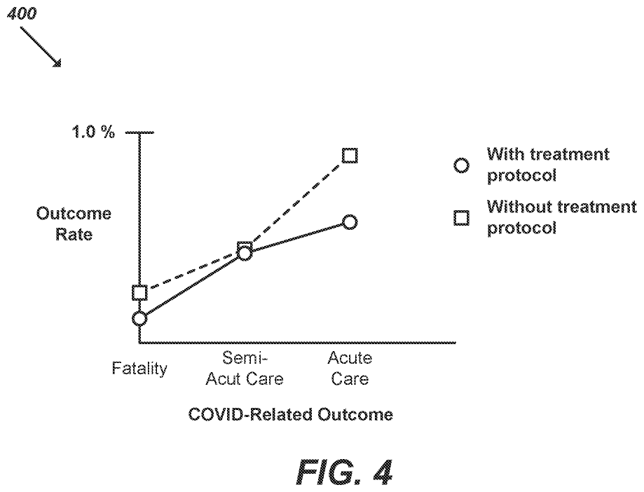

FIG. 4 illustrates a plot 400 of results using a treatment protocol of some embodiments. Table 3, below, shows the data included in plot 400. Various other outcomes (e.g., admission to an intensive care unit) may be associated with such populations and may show similar reduction in negative outcomes.

TABLE 3

| Outcome | Population without treatment | Population with treatment |
|---|---|---|
| Total | 33,000 | 2,600 |
| COVID-Related Fatality | 78 (0.24%) | 3 (0.12%) |
| COVID-Related Semi-Acute | 147 (0.45%) | 11 (0.42%) |
| Care Only | | |
| COVID-Related Inpatient | 294 (0.89%) | 15 (0.58%) |
| Hospital Admission (Acute | | |
| Care) | | |

As shown and illustrated, the population receiving treatment showed significantly reduced rates of various negative outcomes including semi-acute care, acute care (e.g., inpatient care), and fatalities. In this example, the population includes residents across eleven counties in Southern California who were evaluated and/or treated between Jan. 1, 2020 and Aug. 31, 2020.

No element or act used in the present disclosure should be construed as critical or essential unless explicitly described as such. An instance of the use of the term “and,” as used herein, does not necessarily preclude the interpretation that the phrase “and/or” was intended in that instance. Similarly, an instance of the use of the term “or,” as used herein, does not necessarily preclude the interpretation that the phrase “and/or” was intended in that instance. Also, as used herein, the article “a” is intended to include one or more items and may be used interchangeably with the phrase “one or more.” Where only one item is intended, the terms “one,” “single,” “only,” or similar language is used. Further, the phrase “based on” is intended to mean “based, at least in part, on” unless explicitly stated otherwise.

The foregoing relates to illustrative details of exemplary embodiments and modifications may be made without departing from the scope of the disclosure. Even though particular combinations of features are recited in the claims and/or disclosed in the specification, these combinations are not intended to limit the possible implementations of the disclosure. In fact, many of these features may be combined in ways not specifically recited in the claims and/or disclosed in the specification. For instance, although each dependent claim listed below may directly depend on only one other claim, the disclosure of the possible implementations includes each dependent claim in combination with every other claim in the claim set.

We claim:

1. A method of treatment for a subject suffering from an infectious disease, the method comprising:
administering, to the subject, a first dose of an antipsychotic medicament;
waiting a specified delay period; and
administering, to the subject, a second dose of the antipsychotic medicament.

2. The method of claim 1 further comprising, iteratively:
waiting the specified delay period; and
administering, to the subject, an additional dose of the antipsychotic medicament.

3. The method of claim 2, wherein the specified delay period is one day, one week, two weeks, or one month.

4. The method of claim 2, wherein the first dose, the second dose, and each additional dose are subtherapeutic doses.

5. The method of claim 1, wherein the antipsychotic medicament comprises a typical antipsychotic medicament.

6. The method of claim 1, wherein the antipsychotic medicament comprises an atypical antipsychotic medicament.

7. The method of claim 1, wherein the infectious disease is associated with a species of coronavirus or a single-stranded ribonucleic acid (RNA) virus.

8. A method of preventing a subject from contracting an infectious disease, the method comprising:
administering, to the subject, a first dose of an antipsychotic medicament;
waiting a specified delay period; and
administering, to the subject, a second dose of the antipsychotic medicament.

9. The method of claim 8 further comprising, iteratively:
waiting the specified delay period; and
administering, to the subject, an additional dose of the antipsychotic medicament.

10. The method of claim 9, wherein the specified delay period is one day, one week, two weeks, or one month.

11. The method of claim 9, wherein the first dose, the second dose, and each additional dose are subtherapeutic doses.

12. The method of claim 8, wherein the antipsychotic medicament comprises a typical antipsychotic medicament.

13. The method of claim 8, wherein the antipsychotic medicament comprises an atypical antipsychotic medicament.

14. The method of claim 8, wherein the infectious disease is associated with a species of coronavirus or a single-stranded ribonucleic acid (RNA) virus.

15. A method of prophylaxis and treatment for an infectious disease, the method comprising:
administering, to the subject, a first dose of an antipsychotic medicament;
waiting a specified delay period; and
administering, to the subject, a second dose of the antipsychotic medicament.

16. The method of claim 15 further comprising, iteratively:
waiting the specified delay period; and
administering, to the subject, an additional dose of the antipsychotic medicament.

17. The method of claim 16, wherein the specified delay period is one day, one week, two weeks, or one month, and wherein the first dose, the second dose, and each additional dose are subtherapeutic doses.

18. The method of claim 15, wherein the antipsychotic medicament comprises a typical antipsychotic medicament.

19. The method of claim 15, wherein the antipsychotic medicament comprises an atypical antipsychotic medicament.

20. The method of claim 15, wherein the infectious disease is associated with a species of coronavirus or a single-stranded ribonucleic acid (RNA) virus.
